(19) United States
(12) Patent Application Publication
(10) Pub. No.: US 2026/0256703 A1
(43) Pub. Date: Sep. 3, 2026

(54) METHODS FOR FORMATION OF SURFACE FEATURES ON SOFT MATTER MATERIALS THROUGH SUPERCRITICAL FLUID EXCHANGE

(71) Applicant: CORNING INCORPORATED, CORNING, NY (US)

(72) Inventor: Joseph William Mattson, Painted Post, NY (US)

(21) Appl. No.: 18/878,313

(22) PCT Filed: Jun. 19, 2023
(86) PCT No.: PCT/US2023/025661

§ 371 Date: Dec. 23, 2024

Related U.S. Application Data
(60) Provisional application No. 63/357,322, filed on Jun. 30, 2022.

(51) Int. Cl. A61K 9/50

(52) U.S. Cl. CPC A61K 9/5089

(57) ABSTRACT

A method of surface coating a porous, soft matter substance is provided. A porous, soft matter substance comprising an interior and an exterior is provided. Intrinsic or encapsulated chemicals are transferred from the interior of the porous, soft matter substance to the exterior of the porous, soft matter substance through supercritical exchange. The supercritical fluid exchange may comprise supercritical carbon dioxide (CO₂) extraction.

### CROSS-REFERENCE TO RELATED APPLICATIONS

This application claims the benefit of priority under 35 U.S.C. § 119 of U.S. Provisional Application Ser. No. 63/357,322 filed on Jun. 30, 2022, the content of which is relied upon and incorporated herein by reference in its entirety.

### TECHNICAL FIELD

The present disclosure generally relates to hydrogels and particularly relates to surface features of hydrogels.

### BACKGROUND

Providing soft matter, such as hydrogel beads, with microscale and nanoscale features, particularly functionalized features, is highly desirable. Particularly, there is a need for soft matter with such features in the medical and life sciences fields, such as for drug-delivery, as well as in other applications beyond those fields. However, the application of microscale or nanoscale features to hydrogels can be highly complex and may require numerous serial processing stages, thereby lending itself to being time-intensive and introducing additional unit operations to production processes. Therefore, a need exists for a simplified means of producing uniform features on hydrogel beads.

### SUMMARY

As described herein, a soft matter material is provided, along with means of its production. The soft matter material may be a hydrogel bead. Moreover, the soft matter material may have intrinsic surface features produced via supercritical fluid exchange according to methods described herein. For example, materials encapsulated or otherwise trapped in the interior of the soft matter material are transferred to the surface, or exterior of the soft matter material, via supercritical fluid change to create surface features. Such features can be crystalline or polymeric coatings. Moreover, such features can be functionalized in any number of ways. Nonlimiting examples of how the features may be functionalized include being functionalized for augmented cell growth, being magnetized, or being charged.

In conventional methods, the addition of surface features to hydrogel beads generally occurs through the attachment of the features from an external source to the hydrogel surface via some natural affinity or driving source, such as opposite charges or lower entropic state, and often requires a second unit operation. In contrast, the methods disclosed herein create surface features on cross-linked hydrogels by depositing intrinsic chemicals, such as the residual anions from the cross-linking process or encapsulated polymers, onto the surface of the hydrogel.

In an aspect, a method of surface coating a porous, soft matter substance is provided. The method comprises providing a porous, soft matter substance comprising an interior and an exterior; and transferring intrinsic or encapsulated chemicals from the interior of the porous, soft matter substance to the exterior of the porous, soft matter substance through supercritical exchange.

In an embodiment, transferring occurs by solvent extraction.

In an embodiment, the method further comprises functionalizing crystalline features.

In an embodiment, the porous, soft matter substance comprises a hydrogel.

In an embodiment, the intrinsic or encapsulated chemicals comprise polymers.

In an embodiment, the intrinsic or encapsulated chemicals comprise divalent metal salts.

In an embodiment, the intrinsic or encapsulated chemicals comprise alcohol-based solutions.

In an embodiment, the supercritical exchange comprises supercritical carbon dioxide (CO₂) extraction.

In an aspect, a method of creating a drug delivery vehicle is provided. The method comprises encapsulating a functional substance within a porous, soft matter shell; and coating an exterior surface of the porous, soft matter shell by transporting the functional substance from within the porous, soft matter shell to the exterior surface of the porous, soft matter shell through supercritical fluid exchange.

In an embodiment, the porous, soft matter shell is a hydrogel shell.

In an embodiment, the functional substance comprises a soluble drug.

In an embodiment, the supercritical fluid exchange comprises supercritical carbon dioxide (CO₂) extraction.

In an aspect, a method of surface coating a porous, soft matter shell is provided. The method comprises encapsulating a functional substance within a porous, soft matter; and coating an exterior surface of the porous, soft matter by transporting the functional substance from within the porous, soft matter to the exterior surface of the porous, soft matter through supercritical fluid exchange.

In an embodiment, the porous, soft matter comprises an alginate bead.

In an embodiment, the functional substance comprises calcium carbonate.

In an embodiment, the alginate bead is created by crosslinking a solution of sodium alginate in water in a calcium carbonate solution in ethanol.

In an embodiment, the supercritical fluid exchange comprises supercritical carbon dioxide (CO₂) extraction.

In an aspect, a method of surface coating a porous, soft matter is provided. The method comprises encapsulating a functional substance within a porous, soft matter shell; and coating an exterior surface of the porous, soft matter shell by transporting the functional substance from within the porous, soft matter shell to the exterior surface of the porous, soft matter shell through supercritical fluid exchange.

In an embodiment, the porous, soft matter shell is a hydrogel shell.

In an embodiment, the hydrogel shell is created dropwise through crosslinking in a divalent metal bath.

In an embodiment, the functional substance comprises a polymer or oligomer.

In an embodiment, the functional substance comprises an alcohol-based solution.

In an embodiment, the supercritical fluid exchange comprises supercritical carbon dioxide (CO₂) extraction.

### BRIEF DESCRIPTION OF THE DRAWINGS

FIG. 1 is a schematic of an extraction process to develop surface features as described in embodiments herein.

FIG. 2 is a schematic of movement of cation and anion from hydrogel bulk to surface according to an embodiment herein.

FIG. 3 is a schematic of transfer of encapsulated substance from interior to exterior of hydrogel according to an embodiment herein.

FIG. 4 is a scanning electron microscope (SEM) image of “Baseline” DMC bead showing nominally spherical bead profile and smooth bead surface.

FIG. 5A is an SEM image of scCO₂-dried DMC bead showing smooth bead surface and toroidal or “donut like” shape. FIG. 5B is an SEM image of an scCO₂-dried DMC bead. FIG. 5C is an EDX image mapping carbon and calcium in the scCO₂-dried DMC bead of FIG. 5B. Calcium and carbon are relatively homogeneously dispersed throughout the surface of the bead, with a perhaps calcium rich (or carbon deficient) center.

FIG. 6A is an SEM image of an alginate bead showing nominally spherical shape and obvious surface features. FIG. 6B is an SEM image at 700× magnification of surface features on the alginate bead of FIG. 6A, regularly sized and nominally 10 μm in diameter.

FIG. 7A is an SEM image of features observed on alginate bead. FIG. 7B is an EDX map (calcium and carbon) of the features observed on the alginate bead of FIG. 7A. The features appear to be crystalline in structure and calcium rich, particularly when compared to scCO₂-dried DMC beads (c.f.).

FIG. 8 is a schematic of a method of the inside-out coating process to combine the coating and drying stage in functional hydrogel production according to an embodiment.

### DETAILED DESCRIPTION

The present disclosure belongs to the technology area incorporating the continuous production of hydrogels and the implementation of features on the hydrogels. A non-limiting example is directed to cross-linking (referred to as gelation herein) of polysaccharide solutions into gels of discrete shapes and to the formation of features on the surface of the gels, such as regular crystalline features or polymeric coatings.

In existing production methods of hydrogel beads, aqueous polysaccharide solutions are administered dropwise (such as through vibratory nozzle, spinning disk atomization, among others) into a gelation bath composed of divalent metal salts in either water or alcohol. Most generally, the salt is CaCl₂, CaCO₃, or CaSO₄, though other metals cations such as Mg²⁺, Cu²⁺, or Fe²⁺ may be used. The gelation occurs through a model wherein negatively charged regions of the polysaccharide monomers are drawn to the positively charged metal cation. The cation (C⁺) exists in the resultant dimer, trimer, and ultimately polymer hydrogel, while the anion (A⁻) exists in solution (both inside the bath and trapped within the hydrogel). Traditionally, C⁺ and A⁻ are provided in significant excess and large numbers of unbound C⁺ and A⁻ are found in solution. Typical production methods involve a rinse stage to displace as much of the residual aqueous solution in the gel with alcohol, before being sent to a coating stage and ultimately a drying stage. This drying stage drives off the alcohol and a dry, coated hydrogel is left behind.

Microscale and nanoscale features, particularly functionalized features, on soft matter such as hydrogel beads are highly desirable in the life sciences and medical fields (such as drug-delivery), though applications exist beyond these fields due to potentially unique properties, such as the nonlimiting examples of optical or magnetic properties. The application of these features to the gels, however, can be highly complex and require numerous serial processing stages. Disclosed herein are hydrogel beads with surface features and a means of producing uniform features on hydrogel beads.

Traditionally, the application of surface features can be time-intensive and introduce additional unit operations to the production process. When functionalizing the beads, the hydrogel bead would be considered the “core particle”, which would provide the scaffolding of a functionalized layer. The addition of the functionalized layer generally gives the particle a known charge, either positive or negative. A second functionalized layer, with the opposite charge, is then applied to the now-functionalized core particle; the second functionalized layer often has additional functionality resulting in a functionalized hydrogel. Thus, synthesis of a functionalized hydrogel often requires synthesis of the inner core particle, the outer core functional layer, and the surface functional layer, with the serial addition of the latter two to the core particle.

In contrast, methods described herein use unbound ions (C⁺ and A⁻) associated with gelation to form surface features on the hydrogel without the need for additional synthesis or processing beyond the unit operations already involved in production. These features can either be (a) functional features themselves or (b) functionalized through subsequent processes, thus eliminating the need for secondary functionalization (Case (a)) or primary functionalization (Case (b)). Further, as described herein, alcohol-soluble materials can be trapped or otherwise encapsulated within the hydrogel and brought to the surface of the gel to provide value-added functionality during the scCO₂ solvent removal process.

Described herein is a soft polymer material with surface features and a production method thereof. As mentioned above and provided as a nonlimiting example, hydrogel beads are comprised of cross-linked polysaccharide monomers (or similar) that form a polymer network. During the gelation process, the hydrogel bead is nominally 98%+ ionic solution and 2% polymer network, with the solution containing significant amounts of C⁺ and A⁻ (often 5-10% by mass, e.g.). Supercritical carbon dioxide (CO₂) extraction (scCO₂ herein) is leveraged for two important functions: (1) a viable solvent removal (i.e. “drying”) process for the beads and (2) a process by which surface features are added to the bead surface through solvation-deposition or a similar transport process.

While scCO₂ may be a well-established technology for low-temperature solvent removal from porous or fragile solids in solution, the methods provided herein use a scCO₂ process to deposit entrapped chemicals (such as residual C⁺-A⁻ salts) from the interior of the soft matter material to its surface.

In an embodiment, alginate beads are produced by cross-linking a 2% (wt/wt) solution of sodium-alginate in water in an 8% calcium carbonate (CaCO₃) (wt/wt) solution in ethanol. Residual CaCO₃ salts in the hydrogel matrix will be soluble in scCO₂, as will the alcohol from the solution. Solubilized ethanol and residual CaCO₃ salts are drawn from the center of the bead toward the outer surface, where the CaCO₃ salts deposit on the surface. Ethanol is carried away with the scCO₂ and can later be reclaimed. These features, which may exist as crystalline salts on the surface, can be functionalized for a variety of applications. For example, calcium carbonate can be functionalized to optimize drug delivery, and ferrous carbonate (FeCO₃) can increase the efficiency of coal gasification.

In an embodiment, hydrogel shells are created dropwise and used to encapsulate a functional substance (polymer or oligomer, for example) within the gel. Cross linking occurs in a divalent metal bath and the functional substance is retained in the shell of the hydrogel. Supercritical fluid exchange is used to remove the bulk solvent, such as alcohol, and transport the polymer from the interior to the particle exterior. In this non-limiting example, the supercritical fluid exchange process combines two critical processing functions: core particle coating, and particle drying/solvent removal.

In an embodiment, soluble drugs may be encapsulated into small hydrogel drug delivery vehicles through traditional encapsulation methods. The encapsulated drugs can be loaded onto the surface of a drug delivery vehicle though supercritical exchange from the center to the exterior surface. Loaded drug delivery vehicles can then be used for pharmaceutical applications.

As described herein, value-added chemicals, including but not limited to divalent metal salts or alcohol-based solutions, can be trapped or otherwise encapsulated in porous, soft matter such as hydrogels (beads, monoliths, or otherwise) and deposited on the external surface of that hydrogel to provide functional surface features. According to methods described herein, the value-added chemicals are then extracted out from the interior through the surface to settle on the exterior surface through solvent extraction methods, such as use of scCO₂.

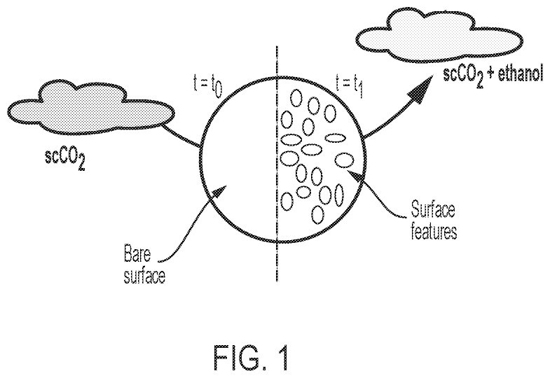

FIG. 1 provides a schematic representing the observations and claims made in this disclosure. Represented in the top of this figure is scCO₂ entering a hydrogel bead with no surface features at time t=t₀. At a later exchange time (nominally tens of minutes, approximately 20 minutes), scCO₂ has extracted the bulk of the ethanol from the bead and has deposited a number of features on surface, usually a divalent metal salt.

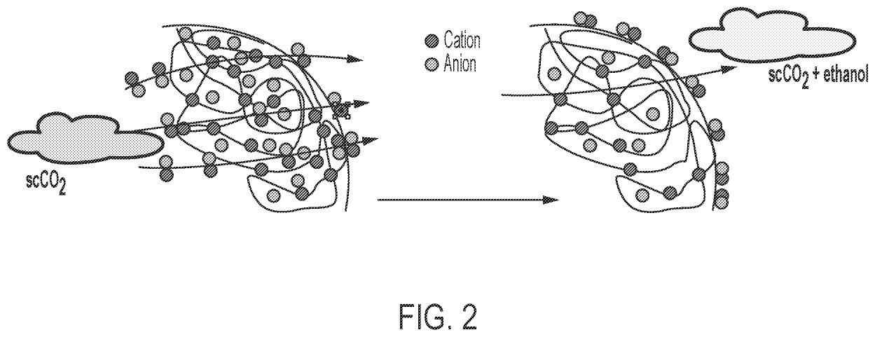

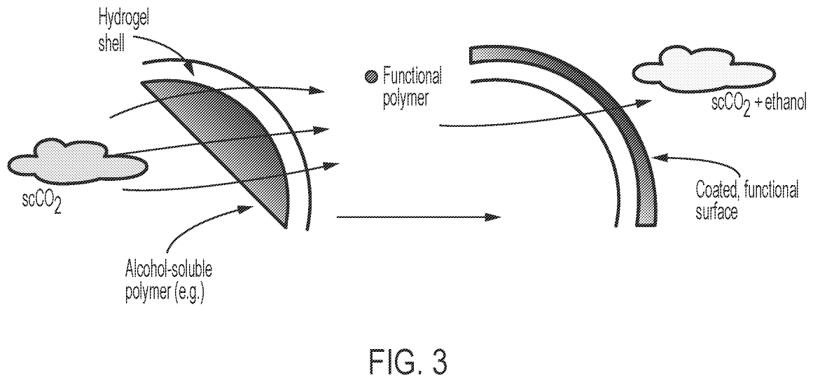

In FIG. 2, a simplified schematic of this process is represented. As cation and anion are solubilized in the alcohol solution, this salt solution is soluble in the scCO₂ solvent. It is assumed that these salts are not extracted from the hydrogel matrix. The exchange process provides two useful functions by combining the coating and drying process into a single, fast operation. In FIG. 3, an alcohol-soluble solution encapsulated in a hydrogel matrix is extracted via scCO₂ extraction (or other solution extraction methods well known to those in the art) and brought to the surface of a hydrogel. Though a portion of a 2D spherical bead is shown, it should be made clear that beads, monoliths, or other hydrogel shapes will exhibit a similar phenomenon and are captured in this disclosure. In FIGS. 2 and 3, the exchange process provides two useful functions by combining the coating and drying process into a single, fast operation.

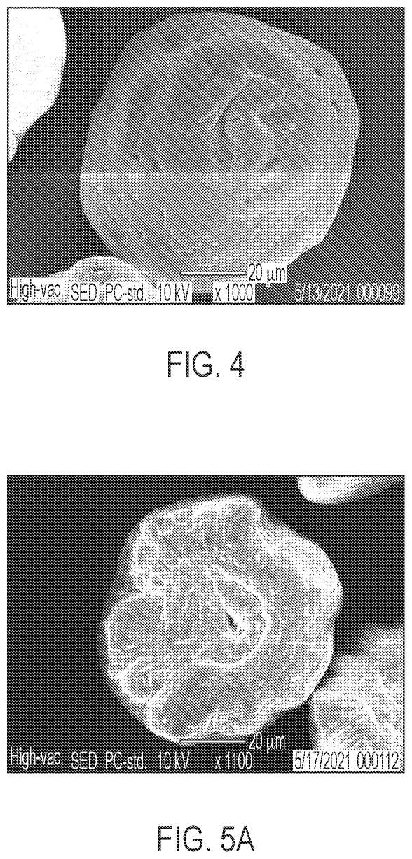

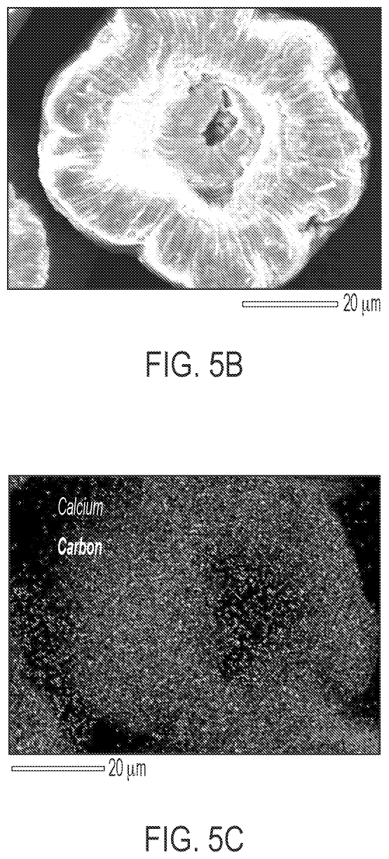

FIG. 4 depicts an SEM image of a DMC bead (made from polygalacutronic acid, PGA, polysaccharide) cross linked in CaCl₂ and dried under a Rotovap to drive off entrained alcohol. Notice that the surface is smooth, and nearly spherical. This image represents that “baseline” or standard performance of a dried DMC bead. FIGS. 5A and 5B show SEM images of same beads rehydrated in water, with the water displaced in alcohol, and the alcohol extracted by scCO₂. Notice the deformation of shape from spherical to toroidal-the origin of this deformation is not understood, though it is reasonable to assume that thermal stresses from the original Rotovap process contributed to the deformation. FIG. 5A shows SEM image of scCO₂-dried DMC bead showing smooth bead surface and toroidal or “donut like” shape. FIG. 5B shows the SEM image and FIG. 5C provides an EDX map of calcium and carbon on a scCO₂-dried DMC bead. Calcium and carbon are relatively homogenously dispersed throughout the surface of the bead or are largely uniformly distributed across the surface, with a calcium rich or carbon deficient “core” at the center of the toroid. Also notice that while surface textures exist, there are no clear features formed on the surface.

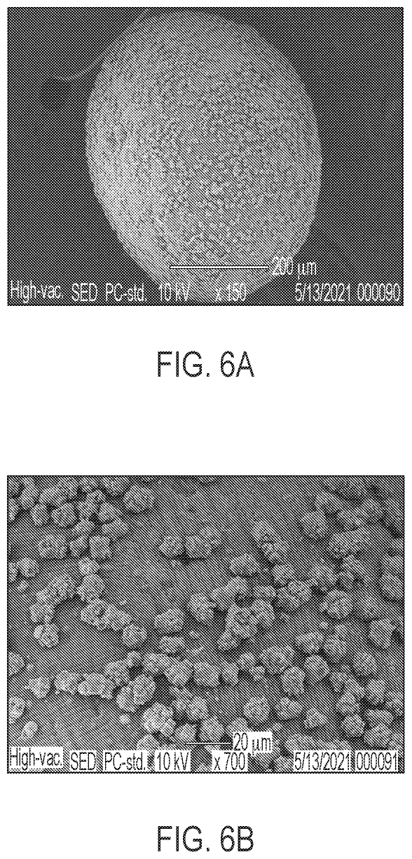

In contrast, FIG. 6A shows a calcium alginate bead (similar to PGA) cross linked in aqueous CaCO₃. These beads were rinsed 3× in alcohol to displace any available water before exposure to supercritical CO₂ extraction. As shown in FIGS. 6B and 6A, the alginate bead, while maintaining a spherical or ellipsoidal shape, is covered in regularly sized “popcorn chicken-like” features uniformly distributed about its surface. These features are assumed to be crystalline salt features. The alginate bead is significantly larger than the DMC bead shown in FIGS. 5A and 5B, which can largely be attributed to difference in manufacturing processes and required specifications. The feature sizes are nominally d~10 μm in diameter.

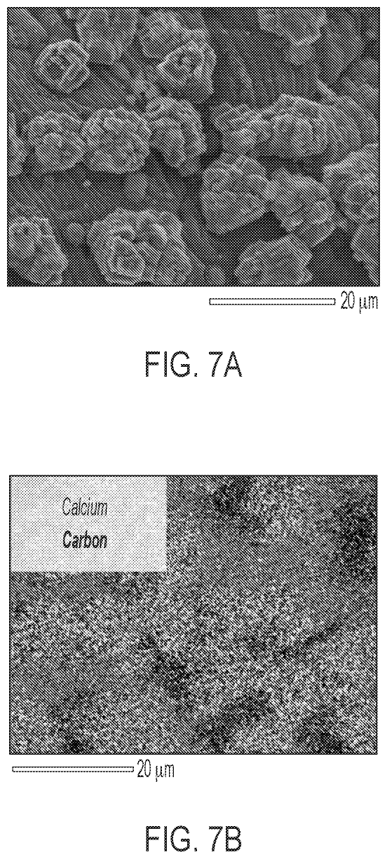

FIGS. 7A and 7B support the hypothesis that these features are crystalline CaCO₃ salts on the surface of the bead. FIG. 7A shows the SEM of features (calcium and carbon) observed on an alginate bead. The features appear to be crystalline in structure and are calcium rich, particularly when compared to scCO₂-dried DMC beads. As shown in FIG. 7B, the EDX map shows calcium rich regions overlapping with the features on the surface, while the bare regions are devoid of calcium and only show a carbon signal. The clear demarcation between calcium and carbon signals and their correlation to the presence of absence of features, couples with the regular size and shape of the features, leads us to believe that these are CaCO₃ features deposited on the surface.

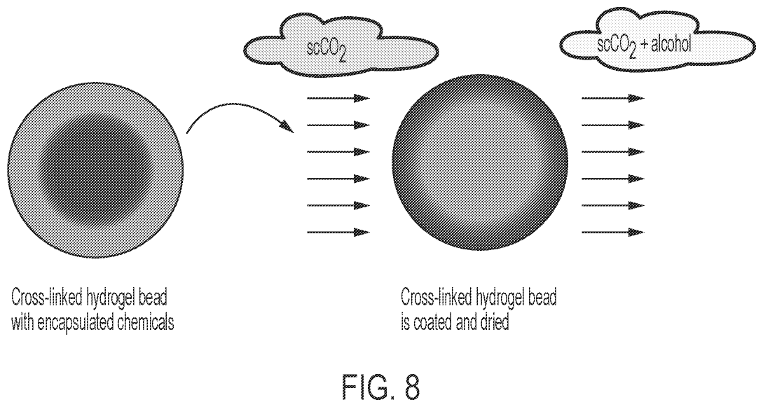

Extrapolation of the phenomena from FIGS. 7A and 7B suggests that a similar observation may be attainable with non-crystalline substances and may not be limited to unbound or excess salt in the gel matrix. The presence of surface features on hydrogels need not be limited to divalent metal salts and may be attainable with polymer or other solutions. This is captured schematically in FIG. 8. In previous solutions, bead formation, coating, and drying occur in (at a minimum) of three serial unit operations (though often many more exist). In the methods disclosed herein, the coating and drying processes occur in parallel by carrying encapsulated value-added chemicals from the interior to the surface of the particle.

In short, this disclosure describes a solvent extraction method to deposit or otherwise transfer intrinsic or encapsulated chemicals from the interior of a porous, soft matter substance to its exterior, either in the form of crystalline features (which may or may not be later functionalized) or in the form of coatings. Thus, the present disclosure is directed to modes of “inside-out coating” of soft matter, such as hydrogels, using solvent extraction practices.

In a first (1) aspect, a method of surface coating a porous, soft matter substance is provided. The method comprises providing a porous, soft matter substance comprising an interior and an exterior; and transferring intrinsic or encapsulated chemicals from the interior of the porous, soft matter substance to the exterior of the porous, soft matter substance through supercritical exchange.

In a second (2) aspect, according to the method of aspect 1, transferring occurs by solvent extraction.

In a third (3) aspect, according to the method of aspects 1-2, the method further comprises functionalizing crystalline features.

In a fourth (4) aspect, according to the method of aspects 1-3, the porous, soft matter substance comprises a hydrogel.

In a fifth (5) aspect, according to the method of aspects 1-4, the intrinsic or encapsulated chemicals comprise polymers.

In a sixth (6) aspect, according to the method of aspects 1-4, the intrinsic or encapsulated chemicals comprise divalent metal salts.

In a seventh (7) aspect, according to the method of aspects 1-4, the intrinsic or encapsulated chemicals comprise alcohol-based solutions.

In an eighth (8) aspect, according to the method of aspects 1-7, the supercritical exchange comprises supercritical carbon dioxide (CO₂) extraction.

In a ninth (9) aspect, a method of creating a drug delivery vehicle is provided. The method comprises encapsulating a functional substance within a porous, soft matter shell; and coating an exterior surface of the porous, soft matter shell by transporting the functional substance from within the porous, soft matter shell to the exterior surface of the porous, soft matter shell through supercritical fluid exchange.

In a tenth (10) aspect, according to the method of aspect 9, the porous, soft matter shell is a hydrogel shell.

In an eleventh (11) aspect, according to the method of aspects 9-10, the functional substance comprises a soluble drug.

In a twelfth (12) aspect, according to the method of aspects 9-11, the supercritical fluid exchange comprises supercritical carbon dioxide (CO₂) extraction.

In a thirteenth (13) aspect, a method of surface coating a porous, soft matter shell is provided. The method comprises encapsulating a functional substance within a porous, soft matter; and coating an exterior surface of the porous, soft matter by transporting the functional substance from within the porous, soft matter to the exterior surface of the porous, soft matter through supercritical fluid exchange.

In a fourteenth (14) aspect, according to the method of aspect 13, the porous, soft matter comprises an alginate bead.

In a fifteenth (15) aspect, according to the method of aspects 13-14, the functional substance comprises calcium carbonate.

In a sixteenth (16) aspect, according to the method of aspects 14-15, the alginate bead is created by crosslinking a solution of sodium alginate in water in a calcium carbonate solution in ethanol.

In a seventeenth (17) aspect, according to the method of aspects 13-16, the supercritical fluid exchange comprises supercritical carbon dioxide (CO₂) extraction.

In an eighteenth (18) aspect, a method of surface coating a porous, soft matter is provided. The method comprises encapsulating a functional substance within a porous, soft matter shell; and coating an exterior surface of the porous, soft matter shell by transporting the functional substance from within the porous, soft matter shell to the exterior surface of the porous, soft matter shell through supercritical fluid exchange.

In a nineteenth (19) aspect, according to the method of aspect 18, the porous, soft matter shell is a hydrogel shell.

In a twentieth (20) aspect, according to the method of aspect 19, the hydrogel shell is created dropwise through crosslinking in a divalent metal bath.

In a twenty-first (21) aspect, according to the method of aspects 18-20, the functional substance comprises a polymer or oligomer.

In a twenty-second (22) aspect, according to the method of aspects 18-20, the functional substance comprises an alcohol-based solution.

In a twenty-third (23) aspect, according to the method of aspects 18-22, the supercritical fluid exchange comprises supercritical carbon dioxide (CO₂) extraction.

It will be appreciated that the various disclosed embodiments may involve particular features, elements, or steps that are described in connection with that particular embodiment. It will also be appreciated that a particular feature, element, or step, although described in relation to one particular embodiment, may be interchanged or combined with alternate embodiments in various non-illustrated combinations or permutations.

It is also to be understood that, as used herein the terms “the,” “a,” or “an,” mean “at least one,” and should not be limited to “only one” unless explicitly indicated to the contrary. Thus, for example, reference to “an opening” includes examples having two or more such “openings” unless the context clearly indicates otherwise.

All scientific and technical terms used herein have meanings commonly used in the art unless otherwise specified. The definitions provided herein are to facilitate understanding of certain terms used frequently herein and are not meant to limit the scope of the present disclosure.

As used herein, “have,” “having,” “include,” “including,” “comprise,” “comprising,” or the like are used in their open-ended sense, and generally mean “including, but not limited to.”

Ranges may be expressed herein as from “about” one particular value, and/or to “about” another particular value. When such a range is expressed, examples include from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

All numerical values expressed herein are to be interpreted as including “about,” whether or not so stated, unless expressly indicated otherwise. It is further understood, however, that each numerical value recited is precisely contemplated as well, regardless of whether it is expressed as “about” that value. Thus, “a dimension less than 10 mm” and “a dimension less than about 10 mm” both include embodiments of “a dimension less than about 10 mm” as well as “a dimension less than 10 mm.”

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that any particular order be inferred.

While various features, elements or steps of particular embodiments may be disclosed using the transitional phrase “comprising,” it is to be understood that alternative embodiments, including those that may be described using the transitional phrases “consisting” or “consisting essentially of,” are implied. Thus, for example, implied alternative embodiments to a method comprising A+B+C include embodiments where a method consists of A+B+C, and embodiments where a method consists essentially of A+B+C.

It will be apparent to those skilled in the art that various modifications and variations can be made to the embodiments described herein without departing from the spirit and scope of the claimed subject matter. Thus, it is intended that the specification cover the modifications and variations of the various embodiments described herein provided such modification and variations come within the scope of the appended claims and their equivalents.

1. A method of surface coating a porous, soft matter substance:
providing a porous, soft matter substance comprising an interior and an exterior; and
transferring intrinsic or encapsulated chemicals from the interior of the porous, soft matter substance to the exterior of the porous, soft matter substance through supercritical fluid exchange.

2. The method of claim 1, wherein transferring occurs by solvent extraction.

3. The method of claim 1, further comprising functionalizing crystalline features.

4. The method of claim 1, wherein the porous, soft matter substance comprises a hydrogel.

5. The method of claim 1, wherein the intrinsic or encapsulated chemicals comprise polymers.

6. The method of claim 1, wherein the intrinsic or encapsulated chemicals comprise divalent metal salts.

7. The method of claim 1, wherein the intrinsic or encapsulated chemicals comprise alcohol-based solutions.

8. The method of claim 1, wherein supercritical exchange comprises supercritical carbon dioxide (CO₂) extraction.

9. A method of creating a drug delivery vehicle comprising:
encapsulating a functional substance within a porous, soft matter shell; and
coating an exterior surface of the porous, soft matter shell by transporting the functional substance from within the porous, soft matter shell to the exterior surface of the porous, soft matter shell through supercritical fluid exchange.

10. The method of claim 9, wherein the porous, soft matter shell is a hydrogel shell.

11. The method of claim 9, wherein the functional substance comprises a soluble drug.

12. The method of claim 9, wherein supercritical fluid exchange comprises supercritical carbon dioxide (CO₂) extraction.

13. A method of surface coating a porous, soft matter shell comprising:
encapsulating a functional substance within a porous, soft matter; and
coating an exterior surface of the porous, soft matter by transporting the functional substance from within the porous, soft matter to the exterior surface of the porous, soft matter through supercritical fluid exchange.

14. The method of claim 13, wherein the porous, soft matter comprises an alginate bead.

15. The method of claim 14, wherein the functional substance comprises calcium carbonate.

16. The method of claim 14, wherein the alginate bead is created by crosslinking a solution of sodium alginate in water in a calcium carbonate solution in ethanol.

17. The method of claim 13, wherein supercritical fluid exchange comprises supercritical carbon dioxide (CO₂) extraction.

18. A method of surface coating a porous, soft matter comprising:
encapsulating a functional substance within a porous, soft matter shell; and
coating an exterior surface of the porous, soft matter shell by transporting the functional substance from within the porous, soft matter shell to the exterior surface of the porous, soft matter shell through supercritical fluid exchange.

19. The method of claim 18, wherein the porous, soft matter shell is a hydrogel shell.

20. The method of claim 19, wherein the hydrogel shell is created dropwise through crosslinking in a divalent metal bath.

21. The method of claim 18, wherein the functional substance comprises a polymer or oligomer.

22. The method of claim 18, wherein the functional substance comprises an alcohol-based solution.

23. The method of claim 18, wherein supercritical fluid exchange comprises supercritical carbon dioxide (CO₂) extraction.
